(19) United States
(12) Patent Application Publication
(10) Pub. No.: US 2007/0038019 A1
(43) Pub. Date: Feb. 15, 2007

(54) Erect vibration ring

(76) Inventor: Chih-Yi Weng, Taipei City (TW)

Correspondence Address: LEONG C LEI, PMB # 1008, 1867 YGNACIO VALLEY ROAD, WALNUT CREEK, CA, 94598, US

(21) Appl. No.: 11/203,166

(22) Filed: Aug. 15, 2005

(51) Int. Cl. A61F 5/00

(52) U.S. Cl. 600/38

(57) ABSTRACT

A disposable erect vibration ring functioning as a sexual aid that is inserted onto the root of a penis to stimulate and massage the vagina and clitoris is comprised of an external part and a vibrator; the external part including a resilient ring integrated with an erect tube; the tube containing the vibrator; the vibrator including a capsule shaped casing, a battery, a minute vibration motor, and an on/off switch; and multiple burr type of feelers being disposed on the front of the top of the tube to further help stimulate the private part of the female.

### BACKGROUND OF THE INVENTION

(a) Technical Field of the Invention

The present invention is related to a disposable erect vibration ring, and more particularly, to a sexual aid that is inserted to the root of a penis to produce stimulation and pleasant sense to the genital organ of the female.

(b) Description of the Prior Art

Though having been referred as a vibration condom as generally available in the market, the vibration condom actually is not for contraception or preventing infection; instead, it is strictly functioning as a sex aid to produce more fun and stimulation during the intercourse. As illustrated in FIG. 1 of the accompanying drawings, the so-called vibration condom is essentially comprised of a ring A made of resilient material that is inserted and fastened to the root of a penis. One side of the ring A is connected to a tube B also made of resilient material. A plastic casing C is inserted into the tube B and the casing C contains a small mercury cell C1, a minute vibration motor C2 and an on/off switch C3. Before having a sex, the ring A is inserted onto the root of the penis while keeping the tube (including the plastic casing C) laterally on top of the penis; and then the switch C3 disposed at the end of the plastic casing is turned on to activate the vibration motor C2 to drive the plastic casing C and the tube B to generate vibration at high frequency. As the penis penetrates into the vagina, the tube B touches the sensitive clitoris to put them subject to the contact and massage of vibration at high frequency to bring the female to feel good and reach climax.

The purpose of the vibration condom is to help the female reaching climax. However, poor design and errors of the vibration condom lead to difficulties in operation and bringing pleasant feeling and climax. The failure to compromise ergonomics explains why those difficulties exist in the prior art. Design of the switch C3 is poor. The user has to bend down to fumble just trying to turn on the switch C3 located at the end of the minute plastic casing C once the ring is inserted onto the root of his penis. It is no surprise that the user could get frustrated in enjoying a good sex. The tube B indicates a tangent lie with the ring A, resulting in that when the tube B contacts the entrance of the vagina, the tube B is laterally disposed to the vertical entrance of the vagina; meaning the chance of the tube to effectively contact and massage the clitoris by vibration is thin, particularly in case of a female having thick and fat external genital organ to prevent any contact of her clitoris. Therefore, the prior art is not ideal when put in practical use.

### SUMMARY OF THE INVENTION

The primary purpose of the present invention is to provide a erect vibration ring that is inserted to the root of a penis to produce stimulation and pleasant sense by effectively massaging the female external genital organ by vibration during intercourse thus to help the female to reach orgasm. To achieve the purpose, the present invention, related to a sexual aid is essentially designed according to the physical structure and sensitivity of human body. It is comprised of a resilient ring, an erect tube integrated with the ring, a capsule shaped casing containing a vibrator including a battery, a minute vibration motor and a start switch to be inserted into the tube, and multiple burr shaped feelers disposed on the front of the top of the tube to stimulate the private part of the female along with the vibrator.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the prior art in use.

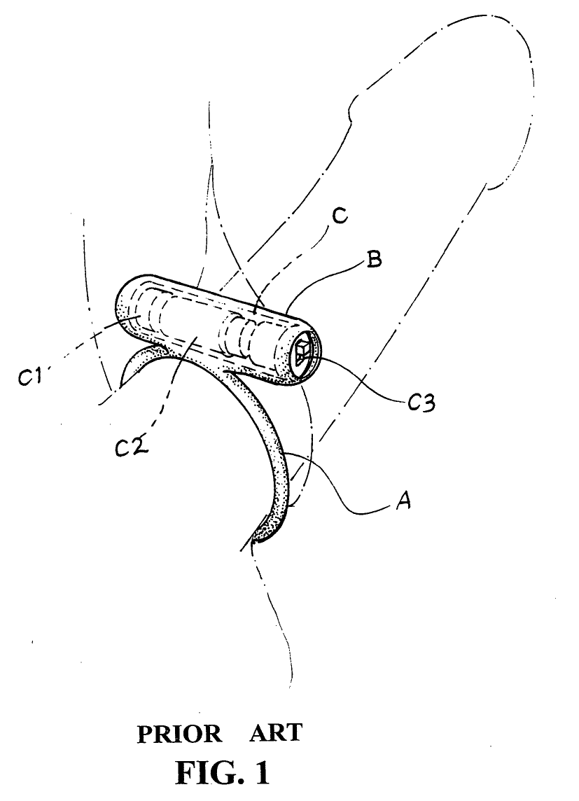

FIG. 2 is an exploded view of the present invention.

FIG. 3 is a sectional view of the present invention.

FIG. 4 is a schematic view showing the present invention in use.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

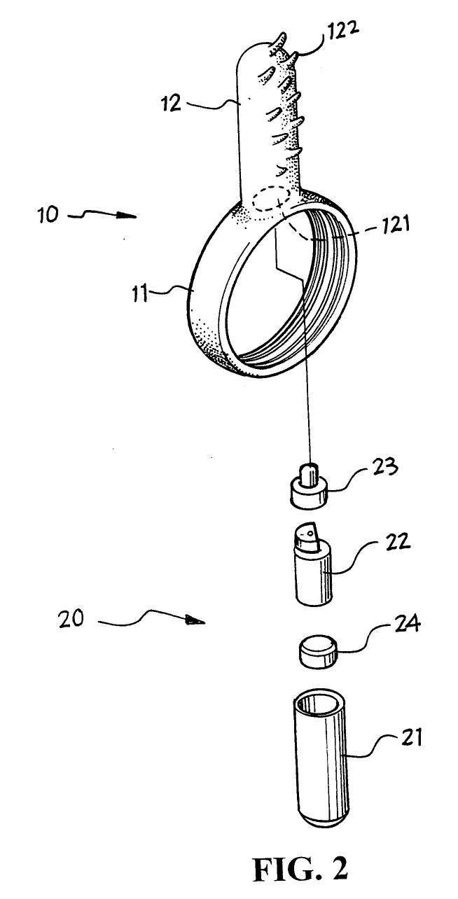

Referring to FIG. 2, the present invention related to a disposal sexual aid made in the form of a ring disposed with an erect tube containing a vibrator is essentially comprised of an external part 10 and a vibrator 20. The external part 10 includes a ring 11 integrated with a tube 12. The ring 11 made of resilient material is intended for insertion onto the root of a penis. The section of the resilient ring 11 indicates flat and the ring 11 is externally connected to the tube 12, which is also made of a resilient material. The axis of the tube 12 is located on the same straight line as the center of the circle of the ring 11. An opening 121 for the tube 12 is disposed at the bottom where the tube 12 is connected to the ring 11. Multiple resilient feelers 122 each in the form of a burr are disposed on the outer surface of the front of the tube 12. A thinner area 123 control by touch is disposed at where behind the top of the tube 12. The bore 120 inside the tube 12 is provided for the insertion of the vibrator 20. The vibrator 20 includes a capsule shape of casing 21 containing a minute vibration motor 22, an on/off switch 23, and a flat mercury cell 24. The on/off switch 23 is located at the top of the vibration motor 22 and exposed out of the surface of the tip of the casing 21.

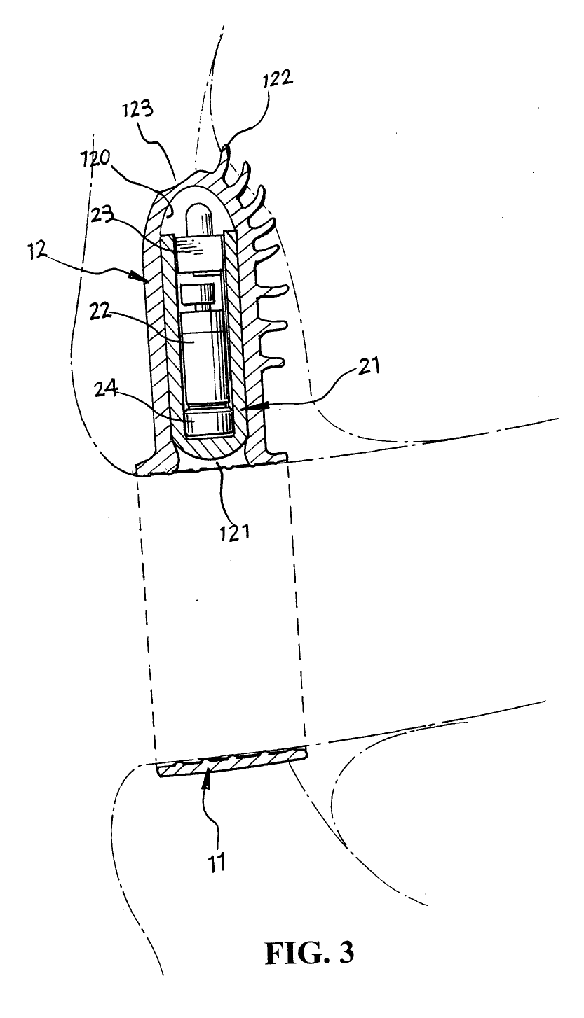

Now referring to FIG. 3, when the vibrator 20 is assembled, put into the bore 120 through the opening 121, and fastened inside the tube 12 due to the resilient material of the tube 12. The on/off switch 23 is placed at where close to the interior of the area 123. To operate the ring 11, simply press the area 123 to activate the vibration motor 22 for both of the vibrator 20 and those fillers 122 to start vibrating.

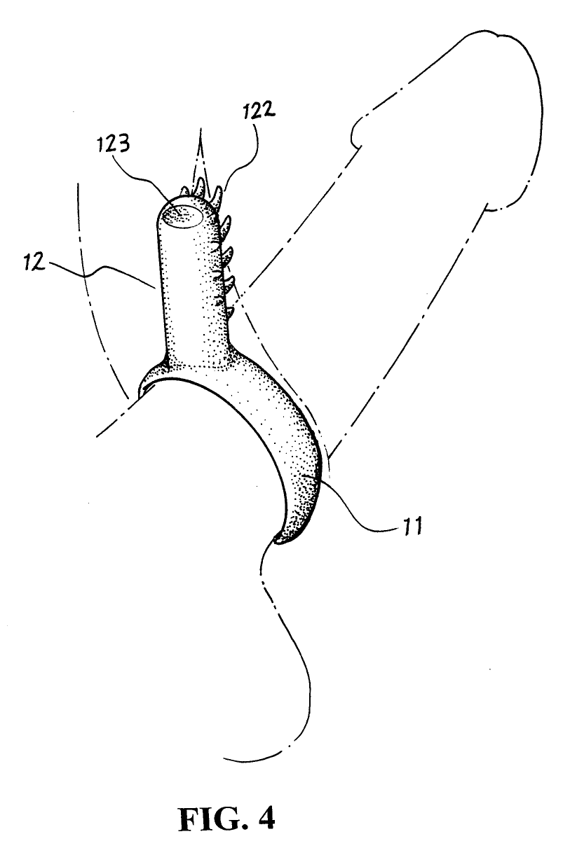

As illustrated in FIG. 4 when the present invention is put in practical use, the erect tube 12 vibrates to effectively touch the local part at the top of the entrance to vagina and the clitoris in vertical relation to the tube 12; meanwhile, the vibration at high frequency of those resilient fillers 122 contact and massage both of the vagina and the clitoris to help the female to reach the status of orgasm.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A disposable erect vibration ring as a sexual aid is comprised of an external part and a vibrator; the external part including a ring made of a resilient material of latex and a tube also made of resilient material; the tube integrated with the ring being disposed at the top of the ring; the tube containing the vibrator to contact and massage a vagina and clitoris of the female; the ring being inserted onto the root of a penis; the section of the ring indicating flat; the axis of the tube and the center of circle of the ring being on the same straight line; an opening being disposed at the bottom where the tube being integrated to the ring; multiple burr shaped resilient fillers being disposed on the outer surface in the front of the tube; the vibrator including a casing; the casing includes a minute vibration motor, an on/off switch, and a cell; and the on/off switch being located at the top of the vibration motor and exposed out of the top surface of the casing.

2. The disposal erect vibration ring of claim 1, wherein a thinner area controlled by touch is located behind the top of the tube; and the area is pressed to activate the vibration motor.

3. The disposal erect vibration ring of claim 1, wherein the on/off switch is related to a minute control switch activated by compression.
